(19) United States
(12) Patent Application Publication
(10) Pub. No.: US 2016/0228011 A1
(43) Pub. Date: Aug. 11, 2016

(54) BIO-INFORMATION ACQUIRING DEVICE AND BIO-INFORMATION ACQUIRING METHOD

(71) Applicant: SHARP KABUSHIKI KAISHA, Osaka-shi, Osaka (JP)

(72) Inventor: Ikuko TSUBAKI, Osaka-shi (JP)

(21) Appl. No.: 15/024,098

(22) PCT Filed: Jul. 8, 2014
(86) PCT No.: PCT/JP2014/068184

§ 371 Date: Mar. 23, 2016

(30) Foreign Application Priority Data
Sep. 26, 2013 (JP) 2013-200445

(51) Int. Cl. A61B 5/024; A61B 5/00

(52) U.S. Cl. CPC A61B 5/024; A61B 5/0077; A61B 5/7278; A61B 5/742; A61B 5/7203

(57) ABSTRACT

A bio-information acquiring device (1) that includes a measurement region setting unit (13) specifying, by image processing, regions respectively corresponding to at least two parts of a living body in frame images forming a moving image obtained by imaging the living body, a pulse wave calculation unit (14) detecting pulse waves in the at least two parts by referring to the specified regions, and a difference calculation unit (15) calculating a phase difference between the detected pulse waves in the at least two parts.

### TECHNICAL FIELD

The present invention relates to a bio-information acquiring device acquiring a pulse wave.

### BACKGROUND ART

A technique is widely used in which a pulse wave is detected by referring to a moving image obtained by imaging a living body (for example, the human body). Here, the “pulse wave” indicates that the pulsation of blood vessels due to ejecting of blood in the heart is expressed as a waveform. Particularly, a pulse wave in which a pressure change of blood vessels is expressed as a waveform is referred to as a “pressure pulse wave”, and a pulse wave in which a volume change of blood vessels is expressed as a waveform is referred to as a “volume pulse wave”.

PTL 1 discloses a method of detecting a volume pulse wave from a face image obtained by imaging the face. In the method disclosed in PTL 1, the volume pulse wave is detected by using a phenomenon in which a person's complexion changes according to a volume change of blood vessels.

In the method disclosed in PTL 1, a dedicated image capturing apparatus is not necessary, and a dedicated illumination device for illuminating a subject (that is, the face of a person to be measured) is not also necessary. Therefore, it is possible to detect pulse waves of a person to be measured by using a general video camera. In the method disclosed in PTL 1, a person to be measured is required to direct his or her face toward a camera, but it is not necessary to restrict body parts (for example, the fingers) of the person to be measured.

Bio-information (an index indicating a physiological condition of a living body) which can be derived from a pulse wave may include pulse wave velocity. Here, the “pulse wave velocity” indicates velocity at which a pulse wave propagates through a blood vessel. The pulse wave velocity may be calculated by dividing the length of a blood vessel between two parts of the living body by a phase difference (difference in arrival time) of pulse waves in the two parts. A pulse wave has the property that propagation velocity thereof increases as a blood vessel is hardened, and thus the pulse wave velocity is used as a useful index for finding cardiovascular diseases such as arteriosclerosis.

PTL 2 discloses an apparatus which calculates a pulse wave velocity on the basis of pulse waves in the base and the tip of the finger. In the apparatus disclosed in PTL 2, pulse waves in the base and the tip of the finger are detected by referring to a finger image obtained by imaging the finger.

In the apparatus disclosed in PTL 2, the finger image is captured by detecting light which is applied from a light source and is transmitted through the finger, by a camera disposed on an opposite side to the light source with respect to the finger. In this case, the finger of a person to be measured is fixed to a predetermined position between the light source and the camera so that images of the base and the tip of the finger are formed in two predefined regions on the finger image (this fixation is realized, for example, by inserting the finger into an insertion hole).

The pulse waves in the base and the tip of the finger are detected as temporal changes in luminance values in the above-described two regions (regions in which the images of the base and tip of the finger are formed) on the finger image. Here, a phenomenon is used in which, if the artery expands, the intensity of light passing through the finger is reduced. The pulse wave velocity is calculated by dividing the length from the base of the finger to the tip thereof by a difference between time points at which a luminance value becomes the minimum in the above-described two regions on the finger image.

### CITATION LIST

### Patent Literature

PTL 1: U.S. Patent No. US2011/0251593A1 (published on Oct. 13, 2011)

PTL 2: Japanese Unexamined Patent Application Publication No. 2008-301915 (published on Dec. 18, 2008)

### SUMMARY OF INVENTION

### Technical Problem

Various bio-information pieces can be derived by using a phase difference of pulse waves in different parts of a living body (for example, the human body). The above-described pulse wave velocity is an example of such bio-information.

However, in a case where a phase difference of pulse waves is calculated by using the apparatus disclosed in PTL 2, it is necessary to form images of two predefined parts (for example, the base and tip of the finger) of a person to be measured in two predefined regions on an image. For this reason, there is a problem in that the living body is required to be restricted so that the parts are fixed to predetermined positions between the light source and the camera.

In the method disclosed in PTL 1, a volume pulse wave is calculated by using a change in a color which is averaged over the entire face of the person to be measured. In other words, the method disclosed in PTL 1 can be said to be a method of detecting a pulse wave in only a single region. Therefore, there is a problem in that an influence of the occurrence of difference in the arrival time of a pulse wave according to each position on the face is not taken into consideration, and a highly accurate measurement result of a pulse wave cannot be obtained.

The present invention has been made in order to solve the above-described problems, and an object thereof is to implement a bio-information acquiring device which can calculate a phase difference of pulse waves in different parts of a living body without restricting the living body, and can derive various bio-information pieces from the phase difference.

### Solution to Problem

In order to solve the problems, according to an aspect of the present invention, there is provided a bio-information acquiring device which derives bio-information from a moving image obtained by imaging a living body, the device including region specifying means for specifying, through image processing, regions respectively corresponding to at least two parts of the living body in frame images forming the moving image; pulse wave detection means for detecting pulse waves in the at least two parts by referring to the regions specified by the region specifying means; and phase difference calculation means for calculating a phase difference between the pulse waves in the at least two parts, detected by the pulse wave detection means.

### Advantageous Effects of Invention

According to the bio-information acquiring device related to an aspect of the present invention, it is possible to achieve an effect in which a phase difference between pulse waves in different parts of a living body can be calculated without restricting the living body.

### BRIEF DESCRIPTION OF DRAWINGS

FIG. 1 is a functional block diagram illustrating a configuration of a bio-information acquiring device according to Embodiment 1 of the present invention.

FIG. 2 Part (a) of FIG. 2 is a diagram illustrating a state in which an imaging section images the face of a person to be measured in Embodiment 1 of the present invention. Part (b) of FIG. 2 is a diagram exemplifying one of a plurality of frame images obtained under an imaging environment illustrated in the part (a) of FIG. 2.

FIG. 3 Part (a) of FIG. 3 is a diagram exemplifying a skin color region extracted from a face region in Embodiment 1 of the present invention. Part (b) of FIG. 3 is a diagram exemplifying two measurement regions in the face region.

FIG. 4 is a flowchart exemplifying a flow of processes of calculating pulse wave velocity in the bio-information acquiring device according to Embodiment 1 of the present invention.

FIG. 5 is a functional block diagram illustrating a configuration of a bio-information acquiring device according to Embodiment 2 of the present invention.

FIG. 6 Part (a) of FIG. 6 is a diagram exemplifying a frame image including a hand region in Embodiment 2 of the present invention. Part (b) of FIG. 6 is a diagram exemplifying two measurement regions in the hand region.

FIG. 7 is a diagram exemplifying calculation points M(i), M(i−1) and M(i+1), vectors u(i) and v(i), and an angle θ in Embodiment 2 of the present invention.

FIG. 8 is a functional block diagram illustrating a configuration of a bio-information acquiring device according to Embodiment 3 of the present invention.

FIG. 9 is a functional block diagram illustrating a configuration of a bio-information acquiring device according to Embodiment 4 of the present invention.

FIG. 10 is a functional block diagram illustrating a configuration of a bio-information acquiring device according to Embodiment 5 of the present invention.

FIG. 11 is a functional block diagram illustrating a configuration of a bio-information acquiring device according to Embodiment 6 of the present invention.

### DESCRIPTION OF EMBODIMENTS

Embodiments of the present invention will be described with reference to the drawings. In the following respective embodiments, a description will be made of a bio-information acquiring device which derives bio-information of a person from a moving image obtained by imaging the person, but the present invention is not limited thereto. In other words, the category of the present invention also includes a bio-information acquiring device which derives bio-information of a living body from a moving image obtained by imaging the living body (any living body having a heart) which is not a person.

### Embodiment 1

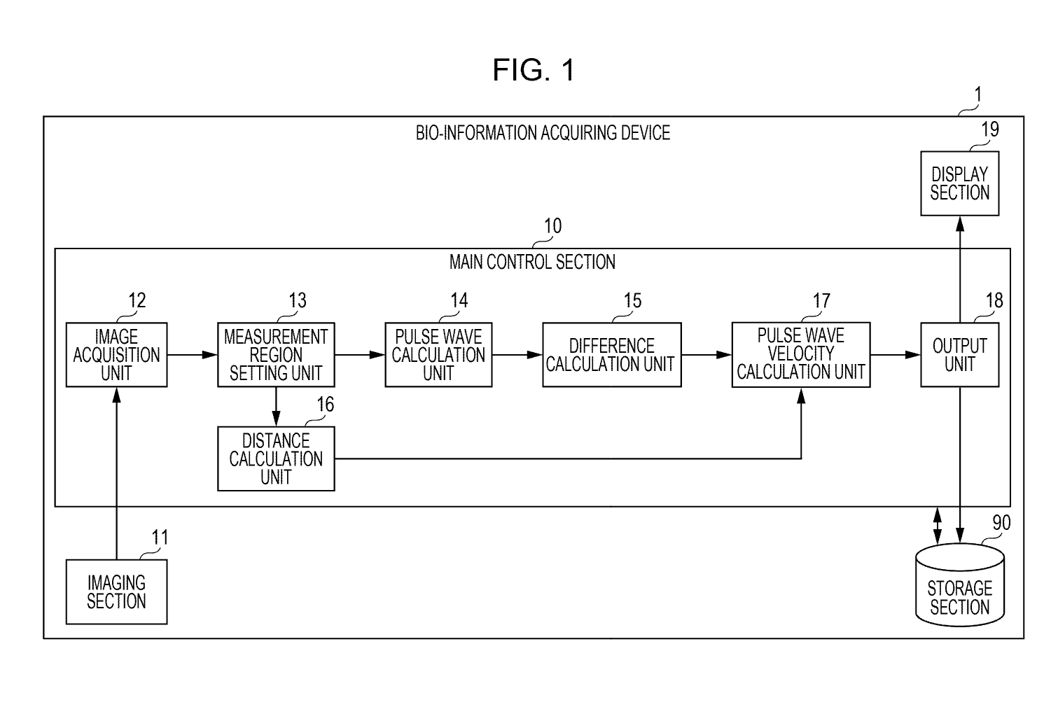

Embodiment 1 of the present invention will be described with reference to FIGS. 1 to 4.

(Bio-Information Acquiring Device 1)

FIG. 1 is a functional block diagram illustrating a configuration of a bio-information acquiring device 1 according to the present embodiment. The bio-information acquiring device 1 includes an imaging section 11, a display section 19, a storage section 90, and a main control section 10.

(Imaging Section 11)

The imaging section 11 generates a moving image by imaging a subject (that is, a person to be measured 121) and sends the generated moving image to an image acquisition unit 12 included in the main control section 10.

Imaging of a subject in the imaging section 11 is performed for a preset measurement time period (for example, 30 seconds). The imaging section 11 may accumulate the moving image for the entire measurement time period and may send the moving image to the image acquisition unit 12, or may divide the moving image at intervals of predetermined time and may sequentially send the moving image to the image acquisition unit 12 in the middle of the measurement time period.

Outputting of the moving image from the imaging section 11 to the image acquisition unit 12 may be performed in a wired manner by using a cable or the like, or may be performed in a wireless manner. The imaging section 11 may record the moving image on a recording medium (for example, a semiconductor memory) provided therein, and the image acquisition unit 12 may read the moving image.

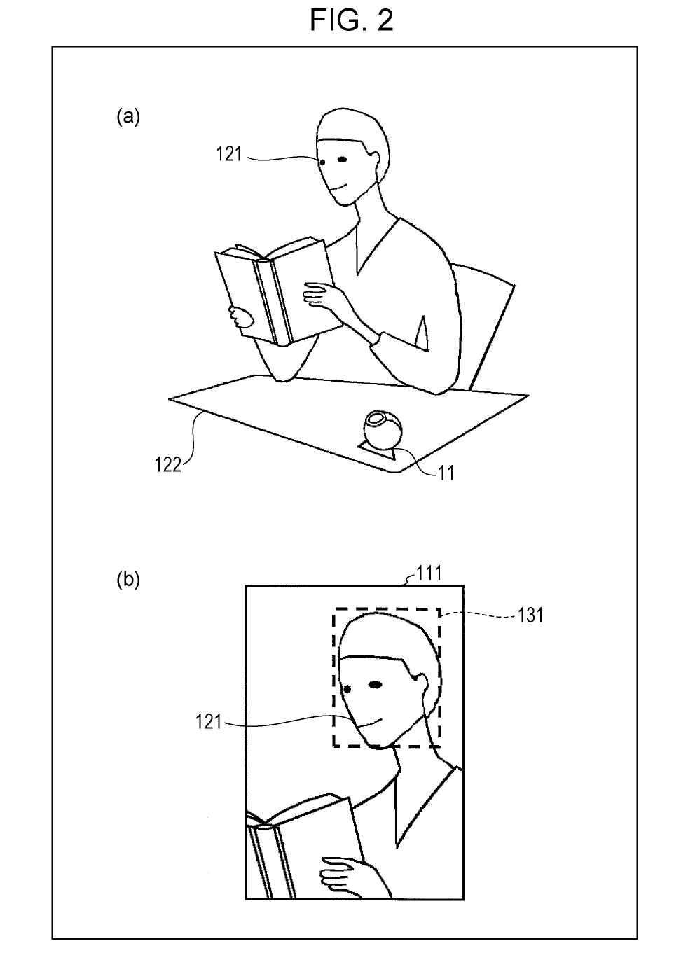

Part (a) of FIG. 2 is a diagram exemplifying a state in which the imaging section 11 images the face of the person to be measured 121. The part (a) of FIG. 2 illustrates a situation in which the imaging section 11 images the person to be measured 121 reading a book, sitting in front of a desk 122. The imaging section 11 is provided on the desk 122 so as to image the face of the person to be measured 121.

As illustrated in the part (a) of FIG. 2, the imaging section 11 can image a body part of the person to be measured 121 without restricting the person to be measured 121. A body part of the person to be measured 121 imaged by the imaging section 11 is not limited to the face. For example, as will be described in Embodiment 2 later, the hand may be imaged as a body part of the person to be measured 121. A luminaire or the like may be provided, and, for example, in relation to a thin part such as the finger, transmitted light from the luminaire or the like may be imaged.

(Display Section 19)

The display section 19 is a display device such as a liquid crystal display. The display section 19 may display the pulse wave velocity calculated by the main control section 10 as data such as image data or text data. Details of an operation of the display section 19 will be described later.

(Storage Section 90)

The storage section 90 is a storage device which stores various programs executed by the main control section 10, and data used by the programs.

(Main Control Section 10)

The main control section 10 generally controls operations of the imaging section 11 and the display section 19. A function of the main control section 10 may be realized by a CPU (central processing unit) executing the programs stored in the storage section 90.

In the present embodiment, the main control section 10 functions as the image acquisition unit 12, a measurement region setting unit 13 (region specifying means), a pulse wave calculation unit 14 (pulse wave detection means), a difference calculation unit 15 (phase difference calculation means), a distance calculation unit 16 (distance calculation means), a pulse wave velocity calculation unit 17 (velocity calculation means), and an output unit 18.

(Image Acquisition Unit 12)

The image acquisition unit 12 decomposes a moving image sent from the imaging section 11 into frames so as to generate frame images. In a case where the generated frame images are coded, the image acquisition unit 12 decodes the frame images. The image acquisition unit 12 sends the frame images to the measurement region setting unit 13.

In a case where the frame images are sent from the imaging section 11 in the unit of a single frame, the process of decomposing a moving image into frames in the image acquisition unit 12 is not necessary.

(Measurement Region Setting Unit 13)

The measurement region setting unit 13 reads the frame images sent from the image acquisition unit 12 and sets a measurement region therein. The measurement region is a region in a frame image, corresponding to a part as a target for detecting a pulse wave among body parts of the person to be measured.

The measurement region is required to be selected from a region in which the skin of the person to be measured is imaged in the frame image. This is because a pulse wave is detected by using temporal changes in a skin color of the person to be measured. The present invention is aimed to measure a pulse wave in a plurality of parts, and thus the measurement region setting unit 13 sets at least two measurement regions.

A pulse wave is generated due to ejecting of blood from the heart, and propagates to a peripheral part along the artery. For this reason, there is the occurrence of difference between times when a pulse wave arrives at the measurement regions whose distances from the heart are different from each other. Therefore, the measurement region setting unit 13 sets a plurality of measurement regions corresponding to a plurality of parts whose distances from the heart are different from each other.

Hereinafter, a description will be made of a case where the measurement region setting unit 13 sets measurement regions in an image of the face of the person to be measured 121. Part (b) of FIG. 2 is a diagram exemplifying one of a plurality of frame images obtained under the imaging environment illustrated in the part (a) of FIG. 2. In the part (b) of FIG. 2, a frame image 111 indicates one of a plurality of frame images.

The measurement region setting unit 13 performs a face detection process on the frame image. The face detection process may be performed according to an appropriate well-known method. As illustrated in the part (b) of FIG. 2, a face region 131 detected through the face detection process is set in an internal region of the frame image 111 including the entire face of the person to be measured 121. The face region 131 has, for example, a rectangular shape including the entire face image of the person to be measured 121.

Next, the measurement region setting unit 13 extracts a skin color region 141 from the face region 131. In other words, the measurement region setting unit 13 converts a color space of the face region 131 (or the frame image 111) into a color space of HSV (hue, saturation, and value). The measurement region setting unit 13 extracts pixels in which values of H (hue), S (saturation), and V (value) are respectively included in predetermined ranges, as the skin color region 141.

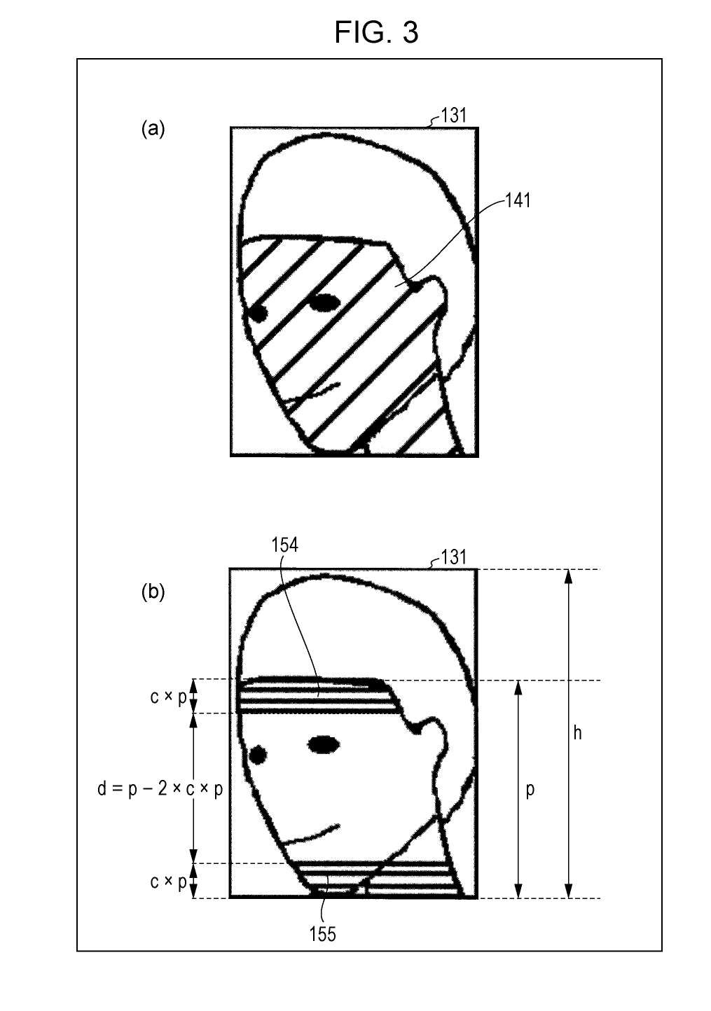

Color spaces other than the HSV color space may be used to extract the skin color region 141. Part (a) of FIG. 3 is a diagram exemplifying the skin color region 141 extracted from the face region 131.

Next, the measurement region setting unit 13 sets two regions including a measurement region 154 (first region) and a measurement region 155 (second region) in the skin color region 141. Here, with reference to the part (b) of FIG. 3, a description will be made of a case where the measurement region 154 corresponding to an upper facial part (a first part, that is, a part which is more distant from the heart of the person to be measured 121) and the measurement region 155 corresponding to a lower facial part (a second part, that is, a position which is closer to the heart of the person to be measured 121) are set.

Part (b) of FIG. 3 is a diagram exemplifying the two measurement regions 154 and 155 in the face region 131. In the part (b) of FIG. 3, a vertical positional relationship is defined by setting a side (that is, a portion close to the head) on which the upper facial part is present as an upper side, and a side (that is, a portion distant from the head) on which the lower facial part is present as a lower side. A direction from the lower side to the upper side (or from the upper side to the lower side) is referred to as a vertical direction.

The measurement region setting unit 13 calculates a skin color region height p. The skin color region height p is an amount obtained as a value of a difference between (i) a coordinate in the vertical direction of a pixel located at an upper end of the skin color region 141 and (ii) a coordinate in the vertical direction of a pixel located at a lower end of the skin color region 141.

Next, the measurement region setting unit 13 calculates a measurement region height c×p by using the skin color region height p and a preset constant c (where 0<c<1).

The measurement region setting unit 13 sets, as the measurement region 154, a portion included in the lower side range from the upper end of the skin color region 141 to c×p in the skin color region 141. The measurement region setting unit 13 sets, as the measurement region 155, a portion included in the upper side range from the lower end of the skin color region 141 to c×p in the skin color region 141.

The measurement region setting unit 13 sends the frame image, the face region 131, and the measurement regions 154 and 155 to the pulse wave calculation unit 14 and the distance calculation unit 16, respectively.

In the measurement region setting unit 13, the constant c used to set the measurement region 154 (first region) and the constant c used to set the measurement region 155 (second region) may be different values.

A method of setting a measurement region in the measurement region setting unit 13 is not limited to the above-described method. For example, a method of detecting the eye and the mouth through a well-known facial organ detection process may be used. In this case, in a frame image, a portion above the eye in the skin color region 141 may be set as the measurement region 154, and a portion under the mouth in the skin color region 141 may be set as the measurement region 155. Even in a case where the face is obliquely imaged, a direction of the face may be further detected in order to appropriately select the vertical direction of the face.

In a frame image, not only upper and lower portions of the face, but also other portions such as left and right portions of the face may be set as measurement regions. The number of measurement regions is not necessarily limited to two, as long as a plurality of measurement regions are set. For example, a portion near the nose detected through a facial organ detection process may be further set as a measurement region. Therefore, the measurement region setting unit 13 may set N (where N is an integer of 2 or greater) measurement regions.

In the present embodiment, a case where a measurement region is selected in each frame is exemplified. On the other hand, a measurement region may be set in an initial frame, and the measurement region set in the initial frame may be used without being changed in subsequent frames. For example, a measurement region may be selected at a constant frame interval such as five frames, and the measurement region set in the previous frame may be used without being changed in other frames.

As another example, a measurement region may be set in an initial frame, and a region corresponding to the measurement region in the previous frame may be set as a measurement region by performing a motion detection process on the previous frame and the present frame in subsequent frames.

(Pulse Wave Calculation Unit 14)

The pulse wave calculation unit 14 detects a pulse wave in each of the measurement regions 154 and 155 set by the measurement region setting unit 13. Computation of a pulse wave in the pulse wave calculation unit 14 is performed by using temporal changes in G (green) values of a color space of RGB (red, green, and blue).

Such a computation method is focused on a property of hemoglobin in blood absorbing green light. Therefore, a pulse wave is computed by approximately regarding a temporal change in a color of a skin surface due to blood flow as a volume pulse wave.

The pulse wave calculation unit 14 calculates an average value of G values of respective pixels inside each measurement region (that is, each of the measurement regions 154 and 155) in each frame image. In a case where a color space of each frame image is not the RGB color space, the pulse wave calculation unit 14 performs conversion into the RGB color space on each frame image in advance.

The pulse wave calculation unit 14 performs a smoothing process using a low-pass filter in a time direction on the average value of the G values so as to remove noise. A frequency characteristic of the low-pass filter is selected so that a frequency of a pulse wave is included in a passband. Therefore, for example, a low-pass filter having a frequency of 4 Hz or lower as a passband is used.

The pulse wave calculation unit 14 performs a normalization process so that a pulse wave has a maximum value of 1 and a minimum value of −1. The normalization process is performed, for example, according to the following Equation (1).

$$\begin{matrix}[ Equation 1 ] & \\ g ( t ) = \frac{2 f ( t ) - max - min}{max - min} & ( 1 )\end{matrix}$$

Here, f(t) on the right side of Equation (1) indicates an average value of G values of the measurement region 154 or 155 after the smoothing process using a low-pass filter is performed. Here, t indicates a frame number. In addition, max indicates the maximum value of f(t) for a measurement time period, and min indicates the minimum value of f(t) for the measurement time period. Further, g(t) on the left side of Equation (1) indicates a pulse wave in the measurement region 154 or 155, obtained through the normalization process.

As a result of a series of processes in the pulse wave calculation unit 14, a pulse wave g1(t) (first pulse wave) in the measurement region 154 and a pulse wave g2(t) (second pulse wave) in the measurement region 155 are detected. The pulse wave calculation unit 14 sends the pulse wave g1(t) and the pulse wave g2(t) to the difference calculation unit 15.

In the pulse wave calculation unit 14, prior to the normalization process, a detrending process for removing a smooth temporal variation may be further performed. An amount used to detect a pulse wave is not limited to a G value. For example, a pulse wave may be detected by performing the same process on luminance of a pixel. Also in a case where the number of measurement regions is three or larger, a pulse wave may be detected in each measurement region in the same manner as in a case of two measurement regions.

(Difference Calculation Unit 15)

The difference calculation unit 15 calculates temporal difference between the pulse wave g1(t) and the pulse wave g2(t), that is, a phase difference between the pulse wave g1(t) and the pulse wave g2(t). The phase difference is calculated by calculating a cross correlation function z(T) between the two pulse wave g1(t) and pulse wave g2(t). Here, τ indicates a shift amount. A shift amount which causes a value of cross correlation function z(T) to become the minimum is calculated as the phase difference.

The cross correlation function z(T) for the pulse wave g1(t) and the pulse wave g2(t) is represented by the following Equation (2). T indicates the number of frames included for a measurement time period.

$$\begin{matrix}[ Equation 2 ] & \\ z ( t ) = \frac{1}{T - τ} {\sum }_{t = 0}^{T - τ - 1} \{ {g}_{1} ( t ) {g}_{2} ( t + τ ) \} & ( 2 )\end{matrix}$$

The difference calculation unit 15 calculates a value of z(τ) in a range of −α≦τ≦α by using a preset constant α. Here, α is the expected maximum value of a phase difference.

The difference calculation unit 15 calculates a value τ=τmin of τ which causes a value of z(τ) to become the minimum in the range of −α≦τ≦α. Here, τmin (frame) indicates a phase difference between the pulse wave g1(t) and the pulse wave g2(t). The difference calculation unit 15 sends a value of the phase difference τmin to the pulse wave velocity calculation unit 17.

The difference calculation unit 15 may calculate the phase difference τmin with decimal pixel accuracy by performing parabola fitting or spline interpolation by using τmin and a value of the cross correlation function z(τ) in the vicinity thereof.

In a case where imaging is performed by an image sensor using a rolling shutter in the imaging section 11, the imaging is performed so that a pixel value of a pixel in a frame image is obtained in a delayed manner as the pixel is located on a lower side therein. In this case, the difference calculation unit 15 may add (q2−q1)×γ×r/n to the phase difference τmin so as to perform correction of an imaging time difference caused by the rolling shutter on the phase difference τmin.

Here, q1 and q2 respectively indicate average values of coordinates in the vertical direction of pixels included in a first region (for example, the measurement region 154) and a second region (for example, the measurement region 155). γ(s) indicates a difference between an imaging time of a pixel in the uppermost row of an image and an imaging time of a pixel in the lowermost row. In addition, r(frame/s) indicates a frame rate of the moving image sent to the image acquisition unit 12. Further, n indicates the number of pixels of the frame image in the vertical direction.

In a case where the number of measurement regions is three or greater, each phase difference τmin may be calculated with respect to two combinations taken from a plurality of measurement regions in the same manner as in a case of two measurement regions. The phase difference τmin may also be referred to as difference τmin.

(Distance Calculation Unit 16)

The distance calculation unit 16 calculates a distance d (pixel) between the measurement regions 154 and 155 in the face region 131 as d=p−2×c×p with respect to an initial frame. The distance calculation unit 16 calculates a height h (pixel) of the face region 131 by using a value of a difference between an upper end coordinate and a lower end coordinate in the vertical direction of the face region 131. The part (b) of FIG. 3 exemplifies d and h.

The distance calculation unit 16 calculates an inter-part distance D (mm) which is a distance between the part corresponding to the measurement region 154 and the part corresponding to the measurement region 155 as D=H×d/h. The distance calculation unit 16 sends a value of the inter-part distance D to the pulse wave velocity calculation unit 17.

H (mm) is a height of the face of the person to be measured 121, measured in advance, or an average height of a person's face. A value of H is recorded in the storage section 90 in advance, and is read by the distance calculation unit 16 as appropriate.

In the present embodiment, the shortest distance between the measurement regions 154 and 155 is used as the distance d, but a method of calculating the distance d is not limited thereto. For example, the longest distance between the measurement regions 154 and 155 may be used as the distance d. A distance between a central point of the measurement region 154 and a central point of the measurement region 155 may be used as the distance d.

An example of obtaining the inter-part distance D in the initial frame has been described, but the present invention is not limited thereto, and the inter-part distance D may be calculated in the last frame or an intermediate frame. The distance d may be calculated in each frame, and the inter-part distance D may be calculated by using an average value thereof. A conversion expression for obtaining the length of a blood vessel from the inter-part distance D may be prepared in advance, and a value of the length of a blood vessel obtained according to the conversion expression may be used as the inter-part distance D.

In a case where the number of measurement regions is three or greater, the inter-part distance D may be calculated in each of two combinations taken from a plurality of measurement regions.

(Pulse Wave Velocity Calculation Unit 17)

The pulse wave velocity calculation unit 17 calculates pulse wave velocity V (mm/s) by using the phase difference τmin calculated in the difference calculation unit 15 and the inter-part distance D calculated in the distance calculation unit 16.

In other words, the pulse wave velocity calculation unit 17 calculates the pulse wave velocity V according to V=D×r/τmin. Here, r (frame/s) is a frame rate of the moving image sent to the image acquisition unit 12. The pulse wave velocity calculation unit 17 sends a value of the pulse wave velocity V to the output unit 18.

In a case where the number of measurement regions is three or greater, the pulse wave velocity V may be calculated in each of two combinations taken from a plurality of measurement regions.

(Output Unit 18)

The output unit 18 outputs the pulse wave velocity V to a device provided outside the main control section 10. For example, the output unit 18 may output the pulse wave velocity V to the display section 19. The output unit 18 may output the pulse wave velocity V to the storage section 90.

The output unit 18 may convert the pulse wave velocity V as appropriate so that the pulse wave velocity is easily processed in an output target device. For example, in a case where the output unit 18 outputs the pulse wave velocity V to the display section 19, the output unit 18 may convert the pulse wave velocity V from numerical value data into text data or image data.

(Flow of Processes of Calculating Pulse Wave Velocity in Bio-Information Acquiring Device 1)

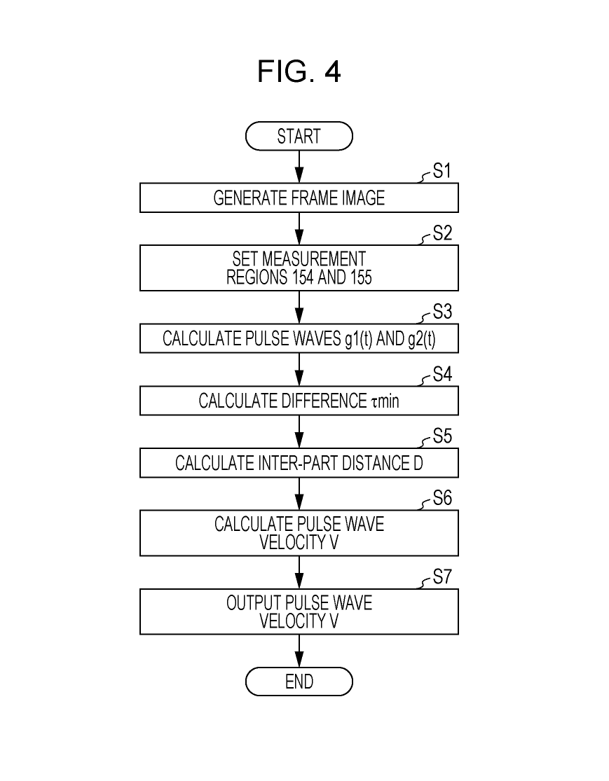

Hereinafter, with reference to FIG. 4, a description will be made of a flow of processes of calculating pulse wave velocity in the bio-information acquiring device 1. FIG. 4 is a flowchart exemplifying a flow of processes of calculating pulse wave velocity in the bio-information acquiring device 1.

First, the image acquisition unit 12 decomposes a moving image sent from the imaging section 11 into frames so as to generate frame images (process S1) (frame image generation step).

The measurement region setting unit 13 sets the two measurement regions 154 and 155 in the frame image (process S2) (region specifying step). The pulse wave calculation unit 14 detects the pulse wave g1(t) in the measurement region 154 and the pulse wave g2(t) in the measurement region 155 (process S3) (pulse wave detection step).

The difference calculation unit 15 calculates the phase difference τmin which is an amount indicating temporal difference between the pulse wave g1(t) and the pulse wave g2(t) (process S4) (phase difference calculation step). The distance calculation unit 16 calculates a distance between a part corresponding to the measurement region 154 and a part corresponding to the measurement region 155, that is, the inter-part distance D (process S5) (distance calculation step).

The pulse wave velocity calculation unit 17 calculates pulse wave velocity V by using the phase difference τmin and the inter-part distance D (process S6) (velocity calculation step). The output unit 18 outputs the pulse wave velocity V to a device (for example, the display section 19 or the storage section 90) provided outside the main control section 10 (process S7) (pulse wave velocity output step).

The pulse wave velocity V is obtained in the bio-information acquiring device 1 through the above-described the processes S1 to S7.

In the above-described example, the pulse wave velocity is output once by using the moving image obtained for a preset measurement time period (for example, 30 seconds), but the present invention is not limited thereto, and the pulse wave velocity may be output at a preset measurement interval (for example, 3 seconds). In this case, a measurement time period and a measurement interval are set in advance, and the pulse wave velocity V is calculated and is output for each measurement interval by using the moving image between a certain time point and a time point before the certain time point by the measurement time period.

(Effects of Bio-Information Acquiring Device 1)

According to the bio-information acquiring device 1, a plurality of measurement regions corresponding to a plurality of parts as pulse wave detection targets can be automatically set through an image recognition process in each frame image of a moving image obtained by imaging the human body of the person to be measured 121.

Even if the person to be measured 121 moves during measurement, regions on the frame image corresponding to a plurality of parts, that is, regions on the frame image which are referred to in order to detect a pulse wave are specified through image processing.

In other words, the bio-information acquiring device 1 can detect the pulse wave g1(t) and the pulse wave g2(t) in the plurality of parts respectively corresponding to the plurality of measurement regions (that is, the measurement regions 154 and 155) even by using images captured without restricting the person to be measured 121.

Therefore, according to the bio-information acquiring device 1, it is possible to achieve an effect in which a plurality of regions for measuring a pulse wave can be set in a captured image of a person to be measured in a simple manner.

According to the bio-information acquiring device 1, it is possible to achieve an effect in which the pulse wave velocity V can be calculated by using the pulse waves g1(t) and g2(t).

### Embodiment 2

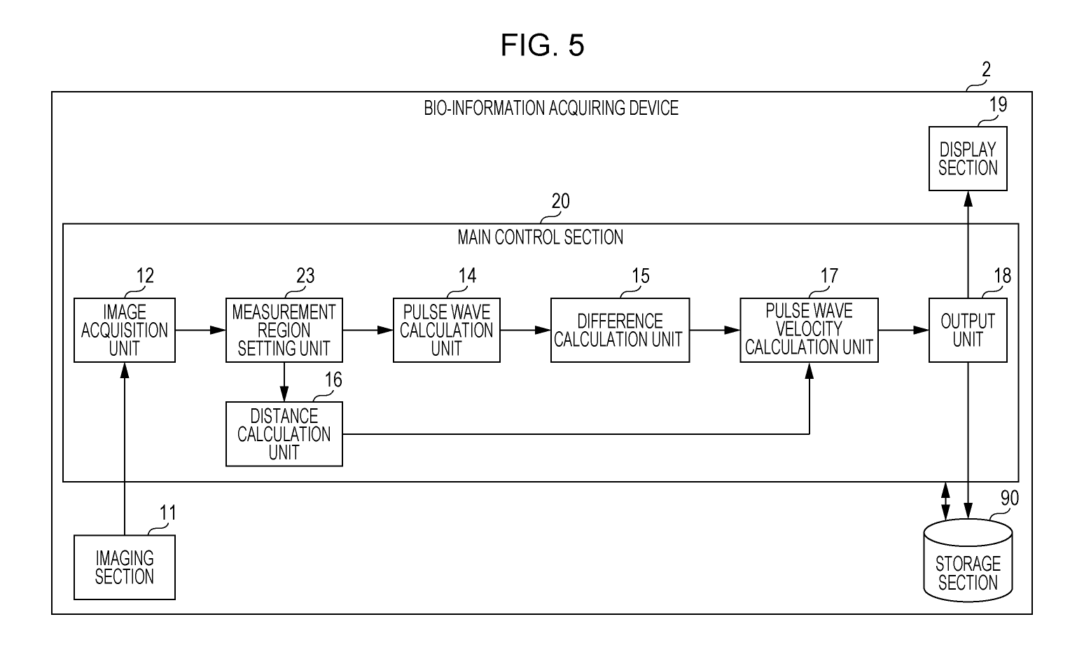

Embodiment 2 of the present invention will be described with reference to FIGS. 5 to 7. For convenience of description, members having the same functions as those of the members described in the above embodiment are given the same reference numerals, and description thereof will be omitted.

(Bio-Information Acquiring Device 2)

FIG. 5 is a functional block diagram illustrating a configuration of a bio-information acquiring device 2 of the present embodiment. The bio-information acquiring device 2 of the present embodiment has a configuration in which (i) the main control section 10 of the bio-information acquiring device 1 of Embodiment 1 is replaced with a main control section 20, and (ii) the measurement region setting unit 13 of the main control section 10 of Embodiment 1 is replaced with a measurement region setting unit 23 (measurement region setting means).

Remaining members of the bio-information acquiring device 2 of the present embodiment are the same as the members of the bio-information acquiring device 1 of Embodiment 1 and are thus given the same reference numerals, and description thereof will be omitted.

(Measurement Region Setting Unit 23)

The measurement region setting unit 23 sets a plurality of measurement regions in the hand of the person to be measured 121. From this viewpoint, the measurement region setting unit 23 of the present embodiment is different from the measurement region setting unit 13 of Embodiment 1 in that a plurality of measurement regions are set in the face of the person to be measured 121.

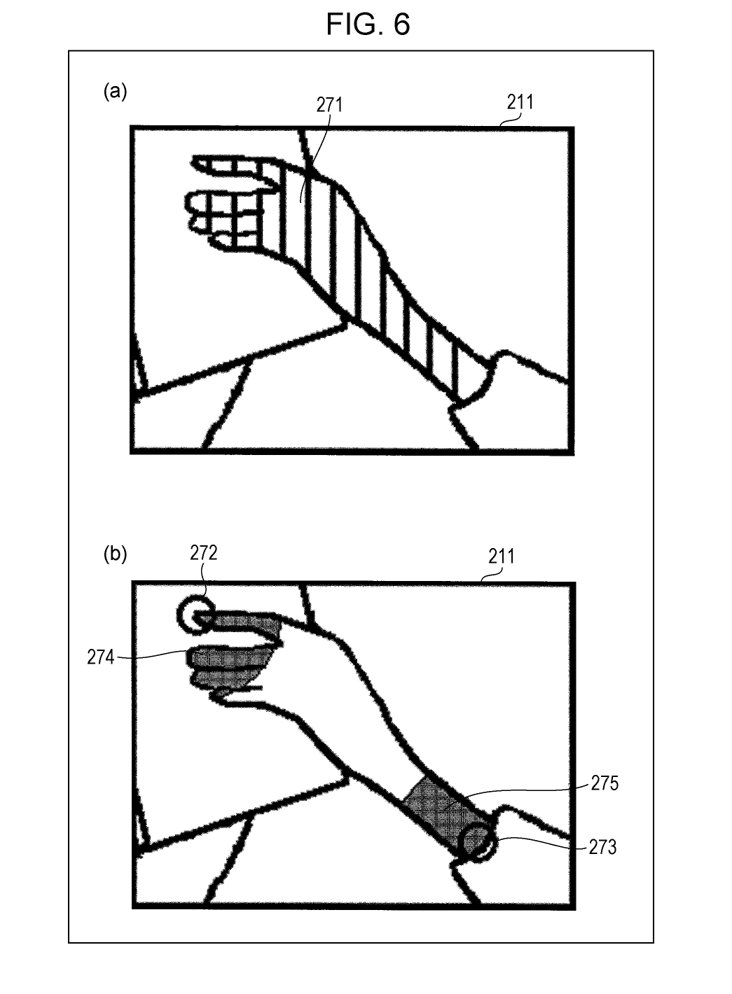

As illustrated in the part (a) of FIG. 2, the imaging section 11 is provided on the desk 122 so as to image the hand of the person to be measured 121. A frame image obtained by imaging the hand of the person to be measured 121 is sent to the measurement region setting unit 23. Part (a) of FIG. 6 is a diagram exemplifying one of a plurality of frame images obtained under the imaging environment illustrated in the part (a) of FIG. 2. In the part (a) of FIG. 6, a frame image 211 indicates one of a plurality of frame images.

The measurement region setting unit 23 performs a hand region detection process on the frame image. The hand region detection process may be performed according to an appropriate well-known method such as extracting a skin color region. A hand region 271 illustrated in the part (a) of FIG. 6 is an example of a region obtained through the hand region detection process.

Next, the measurement region setting unit 23 sets two regions including a measurement region 274 (first region) and a measurement region 275 (second region) in the hand region 271. For example, as illustrated in the part (b) of FIG. 6, a region (that is, a region corresponding to a first part as a part which is more distant from the heart) including the fingertips is set as the measurement region 274, and a region (that is, a region corresponding to a second part as a part which is closer to the heart) including the wrist is set as the measurement region 275.

The part (b) of FIG. 6 is a diagram exemplifying two measurement regions 274 and 275 in the hand region 271. The measurement region 274 is also referred to as a tip side region. The measurement region 275 is also referred to as a root side region.

The measurement region setting unit 23 performs a finger recognition process in order to set the measurement region 274. The finger recognition process may be performed by using an appropriate well-known method but is performed, for example, by using the following method.

In other words, the measurement region setting unit 23 detects, as a tip point, a point which is convex and at which a curved degree of a curve is the maximum in the curve forming a contour of the hand region 271. The tip point may be regarded as a point indicating a fingertip. Hereinafter, a description will be made of an example of a specific process in the measurement region setting unit 23.

First, the measurement region setting unit 23 performs a process of extracting a contour of the hand region 271 and further smoothing a contour shape. Next, the measurement region setting unit 23 sequentially sets a calculation point M(i) (where i=1, 2, . . . ) at a constant interval in a clockwise direction in a curve forming the contour.

Next, the measurement region setting unit 23 calculates a vector u(i) directed from the calculation point M(i) toward a calculation point M(i+1), and a vector v(i) directed from the calculation point M(i) toward a calculation point M(i−1).

The measurement region setting unit 23 calculates an angle θ (where) 0≦θ<360° formed between the vectors u(i) and v(i). If 0≦θ<180°, the calculation point M(i) is located at a convex shape. If 180°<θ<360°, the calculation point M(i) is located at a concave shape.

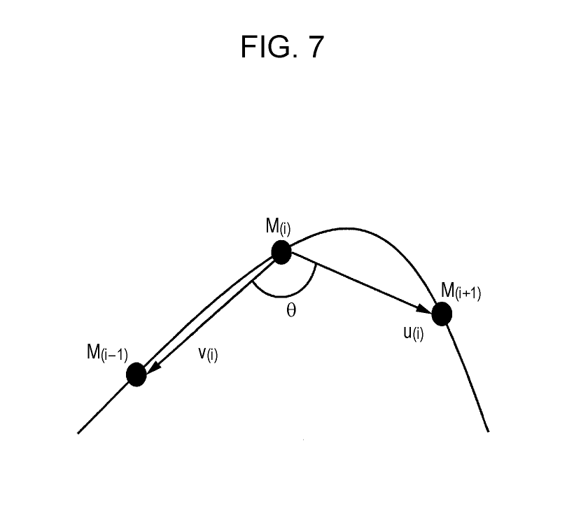

The measurement region setting unit 23 detects the calculation point M(i) in which a value of the angle θ is the minimum, and specifies the calculation point M(i) as a tip point. FIG. 7 exemplifies the calculation points M(i), M(i−1) and M(i+1), the vectors u(i) and v(i), and the angle θ.

As illustrated in the part (b) of FIG. 6, the measurement region setting unit 23 detects a tip point 272 in the hand region 271 as a result of the above-described finger recognition process. The measurement region setting unit 23 detects a point which is longest from the tip point 272 as a root point 273 in the hand region 271.

Successively, the measurement region setting unit 23 sets a region located within a range of a predetermined constant distance from the tip point 272 as the measurement region 274 (that is, a tip side region). The measurement region setting unit 23 sets a region located within a range of a predetermined constant distance from the root point 273 as the measurement region 275 (that is, a root side region).

The measurement region setting unit 23 sends the frame images, the hand region 271, and the measurement regions 274 and 275 to the pulse wave calculation unit 14 and the distance calculation unit 16. Then, in the same manner as in Embodiment 1, the pulse waves g1(t) and g2(t), and the pulse wave velocity V are calculated in the bio-information acquiring device 2.

In a case where the number of measurement regions is three or greater in the measurement region setting unit 23, appropriate regions located in the middle of the measurement region 274 and the measurement region 275 may be added as third and subsequent measurement regions.

The root point 273 is not limited to a point which is most distant from the tip point 272, and may be a point which is separated from the tip point 272 by a predetermined distance or longer.

As a value of H used in the distance calculation unit 16, a size of the hand of the person to be measured 121 measured in advance, or a numerical value indicating an average size of a person's hand (for example, a length from the wrist to a tip of the middle finger) may be used.

As another example of the present embodiment, the imaging section 11 may simultaneously measure both of the face and the hand of the person to be measured 121, the measurement region setting unit 23 may set one or more measurement regions in each of the face and the hand. The difference calculation unit 15 may calculate a phase difference between a pulse wave in the measurement region set in the face and a pulse wave in the measurement region set in the hand. The distance calculation unit 16 may calculate an inter-part distance between the measurement region set in the face and the measurement region set in the hand by using a length between the face and the hand of the person to be measured 121 measured in advance.

The pulse wave velocity calculation unit 17 may calculate pulse wave velocity by using (i) the phase difference between the pulse wave in the measurement region set in the face and the pulse wave in the measurement region set in the hand, and (ii) the inter-part distance between the measurement region set in the face and the measurement region set in the hand.

(Effects of Bio-Information Acquiring Device 2)

According to the bio-information acquiring device 2, a plurality of measurement regions (that is, the measurement regions 274 and 275) can be set in each frame image of a moving image obtained by imaging the hand of the person to be measured 121.

Therefore, also in the bio-information acquiring device 2 of the present embodiment, it is possible to achieve an effect in which the pulse waves g1(t) and g2(t), and the pulse wave velocity V can be calculated without restricting the person to be measured 121 in the same manner as in the bio-information acquiring device 1 of Embodiment 1.

### Embodiment 3

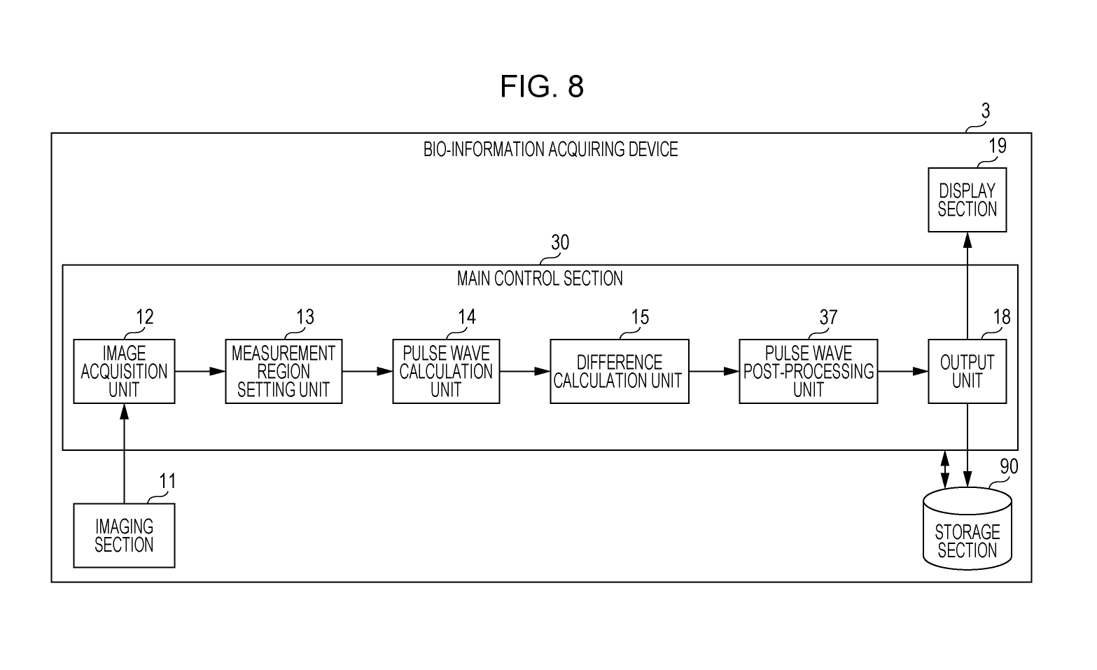

A description will be made of still another embodiment of the present invention with reference to FIG. 8. For convenience of description, members having the same functions as those of the members described in the above embodiments are given the same reference numerals, and description thereof will be omitted.

(Bio-Information Acquiring Device 3)

FIG. 8 is a functional block diagram illustrating a configuration of a bio-information acquiring device 3 of the present embodiment. The bio-information acquiring device 3 of the present embodiment has a configuration in which the main control section 10 of the bio-information acquiring device 1 of Embodiment 1 is replaced with a main control section 30.

Remaining members of the bio-information acquiring device 3 of the present embodiment are the same as the members of the bio-information acquiring device 1 of Embodiment 1 and are thus given the same reference numerals, and description thereof will be omitted.

(Main Control Section 30)

The main control section 30 functions as an image acquisition unit 12, a measurement region setting unit 13, a pulse wave calculation unit 14, a difference calculation unit 15, a pulse wave post-processing unit 37 (pulse wave accuracy increasing means), and an output unit 18.

Therefore, the main control section 30 of the present embodiment has a configuration in which (i) the distance calculation unit 16 is omitted from the main control section 10 of Embodiment 1, and (ii) the pulse wave velocity calculation unit 17 is replaced with the pulse wave post-processing unit 37.

The main control section 30 of the present embodiment is configured to detect a pulse wave with higher accuracy. Therefore, the main control section 30 of the present embodiment is not configured for the purpose of calculating pulse wave velocity unlike the main control section 10 of Embodiment 1.

(Pulse Wave Post-Processing Unit 37)

The pulse wave post-processing unit 37 receives N (where N is an integer of 2 or greater) pulse waves detected in the pulse wave calculation unit 14. Hereinafter, the N pulse waves will be referred to as a pulse wave g1(t) (first pulse wave), a pulse wave g2(t) (second pulse wave), . . . , and a pulse wave gN(t) (N-th pulse wave). N measurement regions set by the measurement region setting unit 13 will be referred to as a measurement region 1A, a measurement region 2A, . . . , and a measurement region NA.

The pulse wave g1(t) indicates a pulse wave calculated in a part corresponding to the measurement region 1A; the pulse wave g2(t) indicates a pulse wave calculated in a part corresponding to the measurement region 2A; and the pulse wave gN(t) indicates a pulse wave calculated in a part corresponding to the measurement region NA.

The pulse wave post-processing unit 37 receives (N−1) phase differences between the measurement region 1A and the remaining measurement regions, calculated in the difference calculation unit 15. Hereinafter, the (N−1) phase differences will be referred to as a phase difference τmin2, a phase difference τmin3, . . . , and a phase difference τminN.

The phase difference τmin2 indicates a phase difference between the pulse wave g1(t) and the pulse wave g2(t); the phase difference τmin3 indicates a phase difference between the pulse wave g1(t) and the pulse wave g3(t); and the phase difference τminN indicates a phase difference between the pulse wave g1(t) and the pulse wave gN(t). Therefore, the phase differences τmin2 to τminN can be said to be respectively phase differences between the pulse wave g1(t) and the pulse waves g2(t) to gN(t).

The pulse wave post-processing unit 37 computes a post-processed pulse wave g(t) according to the following Equation (3).

$$\begin{matrix} [ Equation 3 ] & \\ g ( t ) = \frac{1}{N} \{ {g}_{1} ( t ) + {g}_{2} ( t + τmin2 ) + {g}_{3} ( t + τmin3 ) + … + {g}_{N} ( t + τmin N ) \} & ( 3 )\end{matrix}$$

The post-processed pulse wave g(t) can be said to be an averaged pulse wave obtained by removing the phase differences between the N pulse waves g1(t) to gN(t). It is possible to obtain the post-processed pulse wave g(t) in which an influence of noise components included in the pulse waves g1(t) to gN(t) is reduced by using Equation (3).

A method of computing the post-processed pulse wave g(t) is not limited to Equation (3). For example, phase differences between the N pulse waves g1(t) to gN(t) may be removed, and an average value other than the arithmetic mean (that is, the right side of Equation (3)), such as the weighted mean or the geometric mean may be calculated and be used as the post-processed pulse wave g(t). In addition, phase differences between the N pulse waves g1(t) to gN(t) may be removed, and a statistical value such as a median or a mode may be calculated and be used as the post-processed pulse wave g(t). Further, phase differences between the N pulse waves g1(t) to gN(t) may be removed, and then a component obtained by performing multivariate analysis such as principal component analysis or independent component analysis may be used as the post-processed pulse wave g(t).

The pulse wave post-processing unit 37 sends a value of the post-processed pulse wave g(t) to the output unit 18. The post-processed pulse wave g(t) is output from the output unit 18 to a device provided outside the main control section 30. The same distance calculation unit and pulse wave velocity calculation unit as in Embodiment 1 may be additionally provided, and pulse wave velocity may be further calculated.

(Effect of Bio-Information Acquiring Device 3)

According to the bio-information acquiring device 3, it is possible to achieve an effect in which the post-processed pulse wave g(t) which is a more highly accurate pulse wave can be obtained by respectively detecting the pulse waves g1(t) to gN(t) in a plurality of measurement regions 1A to NA.

### Embodiment 4

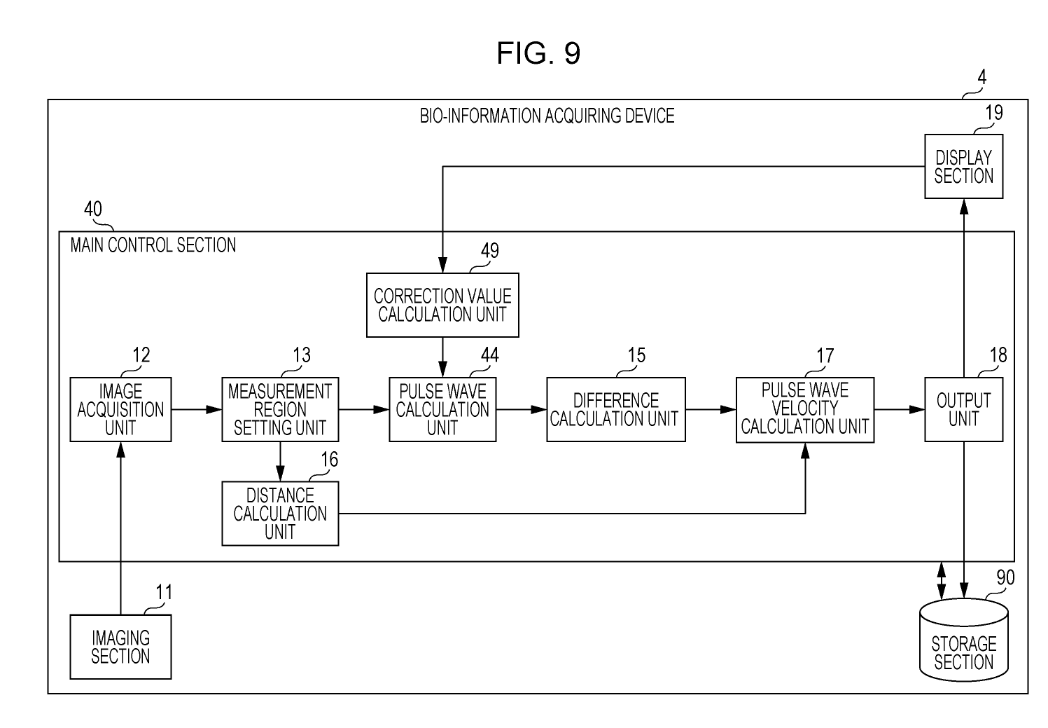

A description will be made of still another embodiment of the present invention with reference to FIG. 9. For convenience of description, members having the same functions as those of the members described in the above embodiments are given the same reference numerals, and description thereof will be omitted.

(Bio-Information Acquiring Device 4)

FIG. 9 is a functional block diagram illustrating a configuration of a bio-information acquiring device 4 of the present embodiment. The bio-information acquiring device 4 of the present embodiment has a configuration in which the main control section 10 of the bio-information acquiring device 1 of Embodiment 1 is replaced with a main control section 40.

Remaining members of the bio-information acquiring device 4 of the present embodiment are the same as the members of the bio-information acquiring device 1 of Embodiment 1 and are thus given the same reference numerals, and description thereof will be omitted.

(Main Control Section 40)

The main control section 40 includes an image acquisition unit 12, a measurement region setting unit 13, a pulse wave calculation unit 44 (pulse wave detection means), a difference calculation unit 15, a distance calculation unit 16, a pulse wave velocity calculation unit 17, a correction value calculation unit 49 (correction value calculation means), and an output unit 18. Therefore, the main control section 40 of the present embodiment has a configuration in which (i) the pulse wave calculation unit 14 of the main control section 10 of Embodiment 1 is replaced with the pulse wave calculation unit 44, and (ii) the correction value calculation unit 49 is additionally provided in the main control section 10 of Embodiment 1.

The main control section 40 of the present embodiment is configured for the purpose of handling a situation in which the imaging section 11 is provided near the display section 19.

For example, a case is assumed in which the face of the person to be measured 121 is directed toward the display section 19. In this case, the face of the person to be measured 121 is irradiated with light emitted from the display section 19. The light emitted from the display section 19 temporally changes according to data (for example, a moving image) displayed on the display section 19. Therefore, a color of a face image of the person to be measured 121 captured by the imaging section 11 temporally changes due to the light emitted from the display section 19 regardless of a blood flow.

Therefore, the main control section 40 of the present embodiment is configured for the purpose of correcting the temporal change in a color of the face image of the person to be measured 121, caused by the light emitted from the display section 19.

(Display Section 19 and Imaging Section 11 of Present Embodiment)

In the present embodiment, the display section 19 outputs a display image to the correction value calculation unit 49 at a predetermined time interval set in advance.

In the present embodiment, the imaging section 11 is disposed on an upper surface of the display section 19, a lower surface of the display section 19, or a side surface of the display section 19. In other words, the imaging section 11 can be said to be disposed near the display section 19. An operation of the imaging section 11 is the same as in Embodiment 1.

(Correction Value Calculation Unit 49)

The correction value calculation unit 49 receives a display image from the display section 19. The correction value calculation unit 49 calculates an average value of G values of respective pixels included in the display image. The average value of the G values may be calculated in the entire display image, and may be calculated in a partial region of the display image. The partial region of the display image is set in advance in the correction value calculation unit 49 prior to calculation of G values.

The correction value calculation unit 49 calculates a correction value by multiplying the average value of the G values by a predetermined constant. The constant for calculating the correction value is set in advance in the correction value calculation unit 49.

The correction value calculated by the correction value calculation unit 49 can be said to be a value for canceling out an influence of light emitted from the display section 19 on a temporal change in a color of a face image of the person to be measured 121. The correction value may be calculated by performing the same process on an average value of luminance of the respective pixels instead of the average value of the G values of the respective pixels.

The correction value calculation unit 49 calculates the above-described correction value in each display image which is sent from the display section 19 at a predetermined time interval. The correction value calculation unit 49 records the correction value calculated at the predetermined time interval in the storage section 90. As a result, time series data of the correction values calculated at the predetermined time interval is obtained.

Successively, the correction value calculation unit 49 performs a process of correcting the time interval of the time series data of the correction values to a time interval at which the imaging section 11 captures a moving image. For example, spline interpolation is used for the correction process.

As a result, the correction value calculation unit 49 calculates a correction value corresponding to each frame image output from the measurement region setting unit 13. The correction value calculation unit 49 sends the correction value corresponding to each frame image to the pulse wave calculation unit 44.

The calculation of the correction value corresponding to each frame image in the correction value calculation unit 49 may be collectively performed after all display images are sent to the correction value calculation unit 49, or may be sequentially performed whenever each display image is sent to the correction value calculation unit 49.

(Pulse Wave Calculation Unit 44)

In the same manner as the pulse wave calculation unit 14 of Embodiment 1, the pulse wave calculation unit 44 calculates an average value of G values of respective pixels inside a measurement region in each frame image. The pulse wave calculation unit 44 calculates a corrected average value of the G values by subtracting the correction value corresponding to each frame image from the average value of the G values of the respective pixels inside the measurement region in each frame image.

The pulse wave calculation unit 44 performs a smoothing process and a normalization process on the corrected average value of the G values so as to detect the pulse waves g1(t) and g2(t) in the same manner as the pulse wave calculation unit 14 of Embodiment 1.

In a case where a correction value is calculated on the basis of an average value of luminance of the respective pixels in the correction value calculation unit 49, the pulse wave calculation unit 44 may detect the pulse waves g1(t) and g2(t) by using the average value of the luminance of the respective pixels inside a measurement region in each frame image.

In the present embodiment, a configuration is exemplified in which the display section 19 is provided alone, but a plurality of display sections may be provided. Therefore, a display section as an output target of the output unit 18 and a display section which sends a display image to the correction value calculation unit 49 may be different from each other.

(Effects of Bio-Information Acquiring Device 4)

According to the bio-information acquiring device 4, it is possible to remove an influence of a temporal change in a color of a face image of the person to be measured 121, caused by light emitted from the display section 19, through correction using a display image which is being displayed on the display section 19.

Therefore, it is possible to achieve an effect in which deterioration in accuracy of a detected pulse wave can be suppressed even in a case where a portion (for example, the face) of the person to be measured 121 which is a target for measuring a pulse wave is irradiated with the light emitted from the display section 19.

In the same manner as the bio-information acquiring device 1 of Embodiment 1, the bio-information acquiring device 4 of the present embodiment is exemplified to have a configuration of calculating the pulse wave velocity V. However, a configuration of the bio-information acquiring device 4 of the present embodiment is not limited thereto, and there may be a configuration in which the post-processed pulse wave g(t) is detected in the same manner as in the bio-information acquiring device 3 of Embodiment 3.

The bio-information acquiring device 4 of the present embodiment may have a configuration in which the hand of the person to be measured 121 is a target part for detecting a pulse wave in the same manner as the bio-information acquiring device 2 of Embodiment 2.

### Embodiment 5

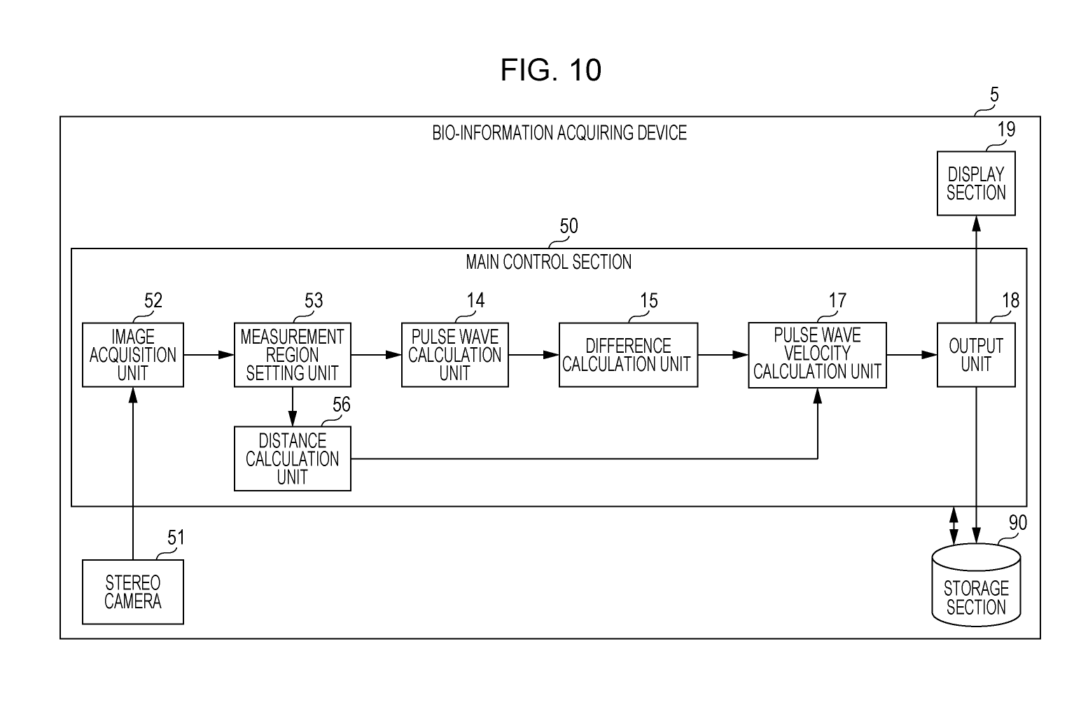

A description will be made of still another embodiment of the present invention with reference to FIG. 10. For convenience of description, members having the same functions as those of the members described in the above embodiments are given the same reference numerals, and description thereof will be omitted.

(Bio-Information Acquiring Device 5)

FIG. 10 is a functional block diagram illustrating a configuration of a bio-information acquiring device 5 of the present embodiment. The bio-information acquiring device 5 of the present embodiment has a configuration in which (i) the imaging section 11 of the bio-information acquiring device 1 of Embodiment 1 is replaced with a stereo camera 51 (imaging section), and (ii) the main control section 10 of the bio-information acquiring device 1 of Embodiment 1 is replaced with a main control section 50.

Remaining members of the bio-information acquiring device 5 of the present embodiment are the same as the members of the bio-information acquiring device 1 of Embodiment 1 and are thus given the same reference numerals, and description thereof will be omitted.

(Stereo Camera 51)

The stereo camera 51 is a camera provided with two lenses including a left eye lens and a right eye lens. The stereo camera 51 images a subject by using the left eye lens and the right eye lens so as to generate a moving image.

Hereinafter, a description will be made of a case where the stereo camera 51 sends a moving image generated by imaging the face of the person to be measured 121 to an image acquisition unit 52 in the same manner as the imaging section 11 of Embodiment 1. The stereo camera 51 may measure parts other than the face of the person to be measured 121, and may image, for example, the hand of the person to be measured 121 in the same manner as the imaging section 11 of Embodiment 2.

(Main Control Section 50)

The main control section 50 includes the image acquisition unit 52, a measurement region setting unit 53 (measurement region setting means), a pulse wave calculation unit 14, a difference calculation unit 15, a distance calculation unit 56 (distance calculation means), a pulse wave velocity calculation unit 17, and an output unit 18. Therefore, the main control section 50 of the present embodiment has a configuration in which the image acquisition unit 12, the measurement region setting unit 13, and the distance calculation unit 16 of the main control section 10 of Embodiment 1 are respectively replaced with the image acquisition unit 52, the measurement region setting unit 53, and the distance calculation unit 56.

(Image Acquisition Unit 52)

The image acquisition unit 52 decomposes a moving image sent from the stereo camera 51 into frames so as to generate a left eye frame image and a right eye frame image. The image acquisition unit 12 sends the left eye frame image and the right eye frame image to the measurement region setting unit 53.

(Measurement Region Setting Unit 53)

The measurement region setting unit 53 reads the left eye frame image and the right eye frame image sent from the image acquisition unit 52. The measurement region setting unit 53 sets a measurement region in one of the left eye frame image (left eye image) and the right eye frame image (right eye image) in the same manner as the measurement region setting unit 13.

Hereinafter, a description will be made of a case where the measurement region setting unit 53 sets two regions including a measurement region 554 (first region) and a measurement region 555 (second region) in the left eye frame image. The measurement region 554 is an upper side region of the face of the person to be measured 121 in the same as the measurement region 154. The measurement region 555 is a lower side region of the face of the person to be measured 121 in the same manner as the measurement region 155.

The measurement region setting unit 53 sends the left eye frame image and the right eye frame image, and the measurement regions 554 and 555 to the pulse wave calculation unit 14 and the distance calculation unit 56.

Operations of the pulse wave calculation unit 14, the difference calculation unit 15, the pulse wave velocity calculation unit 17, and the output unit 18 are the same as those in Embodiment 1, and thus description thereof will be omitted. Hereinafter, a description will be made of an operation of the distance calculation unit 56.

(Distance Calculation Unit 56)

The distance calculation unit 56 calculates disparity (positional difference of each pixel, occurring between the left eye frame image and the right eye frame image) of each of pixels included in the measurement regions 554 and 555 in the left eye frame image by using both of the left eye frame image and the right eye frame image. A method of estimating disparity may employ an appropriate well-known method.

The distance calculation unit 56 calculates an average value of the disparities of the respective pixels included in the measurement region 554 as average disparity δ1 (pixel). The distance calculation unit 56 calculates an average value of the disparities of the respective pixels included in the measurement region 555 as average disparity 62 (pixel).

The distance calculation unit 56 calculates an actual distance K1 (mm) from the subject included in the measurement region 554 to the camera and an actual distance K2 (mm) from the subject included in the measurement region 555 to the camera by using K1=(B×F)/(α×δ1) and K2=(B×F)/(α×δ2), respectively.

Here, B (mm) indicates a base line length of the stereo camera 51, F (mm) indicates a focal length of the stereo camera 51, and α (mm/pixel) indicates a pixel pitch (a horizontal width of one pixel) of the stereo camera 51 in the horizontal direction.

Next, the distance calculation unit 56 calculates an inter-part distance D (mm) which is a distance between a part corresponding to the measurement region 554 and a part corresponding to the measurement region 555 according to the following Equation (4).

[Equation 4]

D=\sqrt{(X₁−X₂)²+(Y₁−Y₂)²+(K₁−K₂)²} (4)

Here, X1, X2, Y1, and Y2 are expressed by the following Equation (5).

$$\begin{matrix}[ Equation 5 ] & \\ \{ \begin{matrix}{X}_{1} = \frac{{K}_{1}}{F} α ( {x}_{1} - \frac{m}{2} ) \\ {X}_{2} = \frac{{K}_{2}}{F} α ( {x}_{2} - \frac{m}{2} ) \\ {Y}_{1} = \frac{{K}_{1}}{F} β ( {y}_{1} - \frac{n}{2} ) \\ {Y}_{2} = \frac{{K}_{2}}{F} β ( {y}_{2} - \frac{n}{2} )\end{matrix} & ( 5 )\end{matrix}$$

β (mm/pixel) indicates a pixel pitch (a vertical width of one pixel) of the left eye frame image in the vertical direction. In addition, m indicates the number of pixels of the left eye frame image in the horizontal direction, and n indicates the number of pixels of the left eye frame image in the vertical direction. (x1,y1) are coordinates indicating a lower end point of the measurement region 554, and (x2,y2) are coordinates indicating an upper end point of the measurement region 555. The coordinates (x1,y1) and (x2,y2) may be calculated in the same manner as in the distance calculation unit 16 of Embodiment 1.

The inter-part distance D calculated by the distance calculation unit 56 of the present embodiment is an amount obtained by taking into consideration a disparity difference (depth difference) between the measurement region 554 and the measurement region 555, and can be said to be an amount which is higher in accuracy than the inter-part distance D calculated by the distance calculation unit 16 of Embodiment 1.

The distance calculation unit 56 sends a value of the inter-part distance D to the pulse wave velocity calculation unit 17. The pulse wave velocity calculation unit 17 can calculate the pulse wave velocity V with higher accuracy by using the value of the inter-part distance D calculated by the distance calculation unit 56 than in Embodiment 1.

The inter-part distance D may not necessarily be calculated by using Equation (4). For example, the inter-part distance D may be calculated by correcting rotation of the stereo camera 51 or an influence of characteristics of the lenses provided in the stereo camera 51.

In a case where the number of measurement regions is three or greater, the inter-part distance D may be calculated in each of two combinations of the measurement regions taken from a plurality of measurement regions.

(Effects of Bio-Information Acquiring Device 5)

According to the bio-information acquiring device 5, it is also possible to calculate the inter-part distance D corresponding to each measurement region in consideration of a disparity difference between the respective measurement regions and by using a moving image captured by the stereo camera 51. Therefore, it is possible to achieve an effect in which the pulse wave velocity V can also be calculated with higher accuracy.

In the same manner as the bio-information acquiring device 1 of Embodiment 1, the bio-information acquiring device 5 of the present embodiment is exemplified to have a configuration in which the hand of the person to be measured 121 is a measurement target. However, a configuration of the bio-information acquiring device 5 of the present embodiment is not limited thereto, and there may be a configuration in which the hand of the person to be measured 121 is a measurement target in the same manner as in the bio-information acquiring device 2 of Embodiment 2.

### Embodiment 6

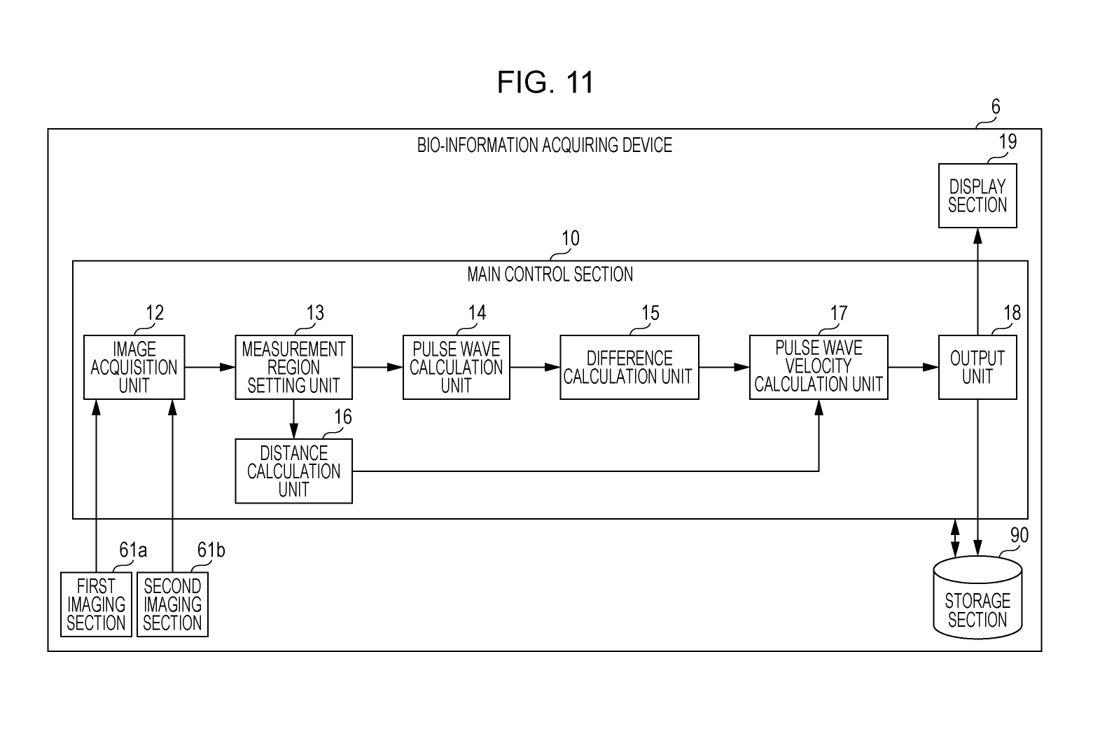

A description will be made of still another embodiment of the present invention with reference to FIG. 11. For convenience of description, members having the same functions as those of the members described in the above embodiments are given the same reference numerals, and description thereof will be omitted.

(Bio-Information Acquiring Device 6)

FIG. 11 is a functional block diagram illustrating a configuration of a bio-information acquiring device 6 of the present embodiment. The bio-information acquiring device 6 of the present embodiment has a configuration in which (i) the imaging section 11 of the bio-information acquiring device 1 of Embodiment 1 is replaced with a first imaging section 61a (imaging section) and a second imaging section 61b (imaging section).

Remaining members of the bio-information acquiring device 6 of the present embodiment are the same as the members of the bio-information acquiring device 1 of Embodiment 1 and are thus given the same reference numerals, and description thereof will be omitted.

A schematic configuration of the bio-information acquiring device 6 of the present embodiment is different from that of the bio-information acquiring device 1 of Embodiment 1 in that a plurality of imaging sections are provided. In the present embodiment, the bio-information acquiring device 6 is exemplified to have a configuration in which the two imaging sections (the first imaging section 61a and the second imaging section 61b) are provided, but the number of imaging sections is not limited to two and may be three or greater.

The first imaging section 61a and the second imaging section 61b respectively image different parts of the person to be measured 121. For example, the first imaging section 61a images the face of the person to be measured 121, and the second imaging section 61b images the fingers of the person to be measured 121.

The first imaging section 61a and the second imaging section 61b output generated moving images to the image acquisition unit 12. The imaging in the first imaging section 61a and the second imaging section 61b are preferably performed in synchronization with each other.

The image acquisition unit 12 decomposes each of the plurality of moving images output from the first imaging section 61a and the second imaging section 61b into frame images.

The measurement region setting unit 13 sets a measurement region in the frame image. As in the present embodiment, in an example in which each of the first imaging section 61a and the second imaging section 61b images the face and the fingers, the measurement region is set in a specific region inside a face region in a frame image of a moving image obtained by imaging the face in the same manner as in Embodiment 1. One or a plurality of measurement regions may be set in the face region.

One or more measurement regions are also set in a frame image of a moving image obtained by imaging the fingers. For example, in a case where the entire image is obtained as a region of the fingers through close imaging, the entire image may be set as a single measurement region. In the above-described manner, in relation to a plurality of moving images, one or more measurement regions are set in each frame image.

The pulse wave calculation unit 14 calculates a pulse wave in each measurement region in the same manner as in Embodiment 1. The difference calculation unit 15 calculates a phase difference between pulse waves calculated in the respective measurement regions for each combination of two measurement regions which can be taken in the same manner as in Embodiment 1. In a case where a plurality of imaging sections are not synchronized with each other, the difference calculation unit 15 also corrects difference between imaging timings.

The distance calculation unit 16 calculates an inter-part distance for each combination of two measurement regions which can be taken by using the respective pulse waves calculated in the measurement regions. In a case where two measurement regions are imaged by different imaging sections, a length of a part of the body of the person to be measured, measured in advance, may be used to calculate an inter-part distance without being changed.

The pulse wave velocity calculation unit 17 calculates pulse wave velocity by using the pulse waves, the phase differences, and the inter-part distance in the same manner as in Embodiment 1. In the same manner as in Embodiment 3, a pulse wave post-processing unit may be provided, and accuracy of a pulse wave may be increased instead of calculating pulse wave velocity.

(Effects of Bio-Information Acquiring Device 6)

According to the bio-information acquiring device 6, it is possible to achieve an effect in which a phase difference between pulse waves can be calculated even in a plurality of parts which are hardly imaged by a single camera. For example, as the first imaging section 61a, an in-camera of a smart phone (that is, a camera mounted on a surface of a side on which a display section of the smart phone is disposed) may be used, and, as the second imaging section 61b, an out-camera of the smart phone (that is, a camera mounted on a surface of the opposite side to the surface on which the in-camera is provided) may be used.

### Modification Example

In the above-described Embodiments 1 to 6, a description has been made of a case where the face or the hand of the person to be measured 121 is a measurement target, but a measurement target is not limited thereto. A measurement target for detecting a pulse wave may be a part in which the skin is exposed among predetermined parts of the body of the person to be measured 121, and may be, for example, the arm, the leg, or the abdomen of the person to be measured 121.

### [Implementations Using Software]

The control blocks (especially, the main control sections 10, 20, 30, 40 and 50) of the bio-information acquiring devices 1, 2, 3, 4, 5 and 6 may be implemented by a logic circuit (hardware) formed on an integrated circuit (IC chip) or the like, and may be implemented by software by using a CPU.

In the latter case, each of the bio-information acquiring devices 1, 2, 3, 4, 5 and 6 includes a CPU executing a command of a program which is software realizing each function, a ROM (read only memory) or a storage device (this is referred to as a “recording medium”) in which the program or various data items are recorded in a computer (or the CPU) readable manner, a RAM (random access memory) in which the program is developed, and the like. The computer (or the CPU) reads the program from the recording medium and executes the program, and thus the object of the present invention is achieved. As the recording medium, a “non-transitory tangible medium”, for example, a tape, a disk, a card, a semiconductor memory, or a programmable logic circuit may be used. The program may be supplied to the computer via any transmission medium (a communication network, a broadcast wave, or the like) which can transmit the program. The present invention can also be implemented in the form of a data signal which is embodied through electronic transmission of the program and is embedded in a carrier.

### CONCLUSION

A bio-information acquiring device (1) related to Aspect 1 of the present invention derives bio-information from a moving image obtained by imaging a living body (for example, the person to be measured 121), and includes region specifying means (measurement region setting unit 13) for specifying, through image processing, regions (for example, the measurement regions 154 and 155) respectively corresponding to at least two parts of the living body in frame images forming the moving image; pulse wave detection means (pulse wave calculation unit 14) for detecting pulse waves (for example, the pulse waves g1(t) and g2(t)) in the at least two parts by referring to the regions specified by the region specifying means; and phase difference calculation means (difference calculation unit 15) for calculating a phase difference (τmin) between the pulse waves in the at least two parts, detected by the pulse wave detection means.

According to the configuration, even if a living body moves during measurement, regions on a frame image corresponding to at least two parts of the living body, that is, regions on the frame image referred to for detecting pulse waves are specified through image processing. Therefore, according to the configuration, it is possible to achieve an effect in which a phase difference between the pulse waves in the at least two parts can be calculated without restricting the living body during measurement.

In the above Aspect 1, the bio-information acquiring device related to Aspect 2 of the present invention may further include distance calculation means (distance calculation unit 16) for calculating an inter-part distance (D) which is a distance between the at least two parts by using a distance (d) between the regions specified by the region specifying means; and velocity calculation means (pulse wave velocity calculation unit 17) for calculating pulse wave velocity (V) by using the phase difference calculated by the phase difference calculation means and the inter-part distance calculated by the distance calculation means.

According to the configuration, it is possible to achieve an effect in which the pulse wave velocity can be calculated without restricting a living body during measurement.

In the above Aspect 1 or 2, the bio-information acquiring device related to Aspect 3 of the present invention may further include pulse wave accuracy increasing means (pulse wave post-processing unit 37) for calculating a statistical value (for example, the post-processed pulse wave g(t)) excluding the phase difference calculated by the phase difference calculation means in at least two pulse waves detected by the pulse wave detection means.

According to the configuration, it is possible to achieve an effect in which a pulse wave with noise reduced and with higher accuracy than in the related art can be calculated without restricting a living body during measurement.

According to the bio-information acquiring device related to Aspect 4 of the present invention, in any one of the above Aspects 1 to 3, the moving image may be obtained as a result of being captured by a plurality of cameras (for example, the first imaging section 61a and the second imaging section 61b).

According to the configuration, it is possible to achieve an effect in which a phase difference between pulse waves can be calculated even in a plurality of parts which are hardly imaged by a single camera.

According to the bio-information acquiring device related to Aspect 5 of the present invention, in any one of the above Aspects 1 to 4, the living body may be a person, the moving image may be obtained by imaging at least one of the face and the hand of the person, and the region specifying means may specify, through image processing, regions (for example, the measurement regions 154 and 155, and the measurement regions 274 and 275) respectively corresponding to at least two parts included in at least one of the face and the hand.

According to the configuration, it is possible to achieve an effect in which accurate pulse wave velocity can be calculated without restricting a living body during measurement by using at least one of, for example, a well-known face detection process and a well-known hand region detection process.

According to the bio-information acquiring device related to Aspect 6 of the present invention, in any one of the above Aspects 1 to 5, the at least two parts may be parts whose distances from the heart of the living body are different from each other.

According to the configuration, it is possible to achieve an effect in which a part suitable for calculating a phase difference between pulse waves can be selected.

In any one of the above Aspects 1 to 6, the bio-information acquiring device related to Aspect 7 of the present invention may further include correction value calculation means (correction value calculation unit 49) for calculating a correction value for canceling out an influence of light emitted from a display section on detection of a pulse wave by referring to an image displayed on the display section (19), and the pulse wave detection means may detect the pulse wave by further using the correction value.

According to the configuration, it is possible to achieve an effect in which a phase difference between pulse waves can be calculated more accurately.

According to the bio-information acquiring device related to Aspect 8 of the present invention, in the above Aspect 2, the moving image may include a left eye image (left eye frame image) and a right eye image (right eye frame image) obtained by imaging the living body with a stereo camera (51), and the distance calculation means may calculate the inter-part distance by further using average disparity (δ1 and δ2) which is calculated by using the left eye image and the right eye image.

According to the configuration, it is possible to achieve an effect in which pulse wave velocity can be calculated more accurately.

In a bio-information acquiring method related to Aspect 9 of the present invention of deriving bio-information from a moving image obtained by imaging a living body, the method includes a region specifying step of specifying, through image processing, regions respectively corresponding to at least two parts of the living body in frame images forming the moving image; a pulse wave detection step of detecting pulse waves in the at least two parts by referring to the regions specified in the region specifying step; and a phase difference calculation step of calculating a phase difference between the pulse waves in the at least two parts, detected in the pulse wave detection step.

According to the configuration, it is possible to achieve an effect in which a phase difference between the pulse waves in the at least two parts can be calculated without restricting the living body during measurement in the same manner as in the bio-information acquiring device related to Aspect 1.

The bio-information acquiring device related to each aspect of the present invention may be implemented by a computer. In this case, the category of the present invention also includes a control program for the bio-information acquiring device which causes the bio-information acquiring device to be implemented by the computer by causing the computer to be operated as each piece of means included in the bio-information acquiring device, and a computer readable recording medium recording the program thereon.

### APPENDIXES

The present invention is not limited to the respective above-described embodiments and may be variously modified within the scope disclosed in the claims, and embodiments obtained by combining the disclosed technical means with other embodiments as appropriate are also included in the technical scope of the present invention. A new technical feature may be formed by combining the pieces of technical means disclosed in the respective embodiments with each other.

The present invention may also be expressed as follows.

In other words, a bio-information acquiring device related to an aspect of the present invention calculates a pulse wave from an image, and includes measurement region setting means for setting at least two measurement regions for calculating the pulse wave; pulse wave detection means for calculating a pulse wave in each measurement region; and difference calculation means for calculating difference between the pulse waves obtained by the pulse wave detection means.

The bio-information acquiring device related to the aspect of the present invention further includes distance calculation means for calculating a distance between the measurement regions; and pulse wave velocity calculation means for calculating pulse wave velocity on the basis of the difference and the distance between the measurement regions.

In the bio-information acquiring device related to the aspect of the present invention, the image includes a face image of a person in whom a pulse wave is measured, and an entire measurement region setting unit sets at least two regions among regions of the face image of the person to be measured as the measurement regions.

In the bio-information acquiring device related to the aspect of the present invention, the image includes a hand image of a person in whom a pulse wave is measured, and an entire measurement region setting unit sets at least two regions among regions of the hand image of the person to be measured as the measurement regions.

The bio-information acquiring device related to the aspect of the present invention further includes pulse wave post-processing means for improving accuracy of the pulse waves by using difference between the pulse waves.

The bio-information acquiring device related to the aspect of the present invention further includes display means for displaying an image, and correction value calculation means for calculating a correction value on the basis of the image displayed by the display means, and the pulse wave detection means calculates the pulse wave by using the correction value.

In the bio-information acquiring device related to the aspect of the present invention, the image obtained by imaging the person to be measured is captured by a stereo camera, and the distance calculation means calculates the distance between the measurement regions by using a depth difference between the measurement regions.

### INDUSTRIAL APPLICABILITY

The present invention may be used for a bio-information acquiring device, particularly, a device measuring a pulse wave.

### REFERENCE SIGNS LIST

1, 2, 3, 4, 5, 6 BIO-INFORMATION ACQUIRING DEVICE
11 IMAGING SECTION
13, 23, 53 MEASUREMENT REGION SETTING UNIT (REGION SPECIFYING MEANS)
14, 44 PULSE WAVE CALCULATION UNIT (PULSE WAVE DETECTION MEANS)
15 DIFFERENCE CALCULATION UNIT (PHASE DIFFERENCE CALCULATION MEANS)
16, 56 DISTANCE CALCULATION UNIT (DISTANCE CALCULATION MEANS)
17 PULSE WAVE VELOCITY CALCULATION UNIT (VELOCITY CALCULATION MEANS)
19 DISPLAY SECTION
37 PULSE WAVE POST-PROCESSING UNIT (PULSE WAVE ACCURACY INCREASING MEANS)
49 CORRECTION VALUE CALCULATION UNIT (CORRECTION VALUE CALCULATION MEANS)
51 STEREO CAMERA (IMAGING SECTION)
61a FIRST IMAGING SECTION (IMAGING SECTION)
61b SECOND IMAGING SECTION (IMAGING SECTION)
121 PERSON TO BE MEASURED
154, 155, 274, 275, 554, 555 MEASUREMENT REGION
D INTER-PART DISTANCE
N INTEGER (INTEGER INDICATING NUMBER OF MEASUREMENT REGIONS)
1A TO NA MEASUREMENT REGION
g1(t) TO gN(t) PULSE WAVE
g(t) POST-PROCESSED PULSE WAVE (STATISTICAL VALUE OF PULSE WAVE)
τmin, τmin2 TO τminN PHASE DIFFERENCE
δ1, δ2 AVERAGE DISPARITY
V PULSE WAVE VELOCITY

1. A bio-information acquiring device which derives bio-information from a moving image obtained by imaging a living body, the device comprising:
region specifying means for specifying, through image processing, regions respectively corresponding to at least two parts of the living body in frame images forming the moving image, the moving image being obtained as a result of being captured by a plurality of cameras;
pulse wave detection means for detecting pulse waves in the at least two parts by referring to the regions specified by the region specifying means;
phase difference calculation means for calculating a phase difference between the pulse waves in the at least two parts, detected by the pulse wave detection means;
distance calculation means for calculating an inter-part distance which is a distance between the at least two parts by using a distance between the regions specified by the region specifying means; and
velocity calculation means for calculating pulse wave velocity by using the phase difference calculated by the phase difference calculation means and the inter-part distance calculated by the distance calculation means.

2-6. (canceled)

7. The bio-information acquiring device according to claim 1, further comprising:
a display section; and
correction value calculation means for calculating a correction value for canceling out an influence of light emitted from the display section on detection of a pulse wave by referring to an image displayed on the display section,
wherein the pulse wave detection means detects the pulse wave by further using the correction value.

8. The bio-information acquiring device according to claim 1,
wherein the moving image includes a left eye image and a right eye image obtained by imaging the living body with a stereo camera, and
wherein the distance calculation means calculates the inter-part distance by further using disparity which is calculated by using the left eye image and the right eye image.

9. A bio-information acquiring method of deriving bio-information from a moving image obtained by imaging a living body, the method comprising:
a region specifying step of specifying, through image processing, regions respectively corresponding to at least two parts of the living body in frame images forming the moving image, the moving image being obtained as a result of being captured by a plurality of cameras;
a pulse wave detection step of detecting pulse waves in the at least two parts by referring to the regions specified in the region specifying step;
a phase difference calculation step of calculating a phase difference between the pulse waves in the at least two parts, detected in the pulse wave detection step;
a distance calculation step of calculating an inter-part distance which is a distance between the at least two parts by using a distance between the regions specified by the region specifying step; and
a velocity calculation step of calculating pulse wave velocity by using the phase difference calculated by the phase difference calculation step and the inter-part distance calculated by the distance calculation step.
